(19) United States
(12) Patent Application Publication
(10) Pub. No.: US 2023/0372589 A1
(43) Pub. Date: Nov. 23, 2023

(54) INTERPOSITIONAL IMPLANT FOR GROWTH PLATE INJURY

(71) Applicant: University of Pittsburgh - Of the Commonwealth System of Higher Education, Pittsburgh, PA (US)

(72) Inventors: Juan M. Taboas, Pittsburgh, PA (US); Jingming Chen, Chicago, IL (US)

(73) Assignee: University of Pittsburgh - Of the Commonwealth System of Higher Education, Pittsburgh, PA (US)

(21) Appl. No.: 18/027,575

(22) PCT Filed: Sep. 20, 2021
(86) PCT No.: PCT/US2021/051096

§ 371 Date: Mar. 21, 2023

Related U.S. Application Data
(60) Provisional application No. 63/080,890, filed on Sep. 21, 2020.

(51) Int. Cl. A61L 27/54; A61L 27/52; A61L 33/00; A61L 27/22; A61L 27/18; A61L 27/26; A61L 27/38

(52) U.S. Cl. CPC A61L 27/54; A61L 27/52; A61L 33/0011; A61L 27/222; A61L 27/18; A61L 27/26; A61L 27/3834; A61L 2400/06; A61L 2400/02; A61L 2300/414; A61L 2430/02

(57) ABSTRACT

Disclosed herein implantable material and methods for treatment of growth plate injuries and other purposes. These materials can be particularly useful for treating children whose growth plates are active, and can help encourage proper healing and inhibit unwanted bone formations. Exemplary compositions can comprise poly (ethylene glycol) (“PEG”), gelatin (“GEL”), and heparin (“HEP”). The PEG, GEL, and HEP components can be present in various forms of these materials, such as methacrylated forms, etc. The implanted materials can be anti-osteogenic and/or ant-mineralization, and can help prevent unwanted bone growth in the implanted area, such as boney tethers, which can inhibit desirable growth plate healing and overall bone growth.

### CROSS-REFERENCE TO RELATED APPLICATIONS

This application claims the benefit of U.S. Provisional Patent Application No. 63/091,824, filed on Oct. 14, 2020, which is incorporated by reference herein in its entirety.

### ACKNOWLEDGMENT OF GOVERNMENT SUPPORT

This invention was made with government support under AR062598 awarded by the National Institutes of Health. The government has certain rights in the invention.

### FIELD

This disclosure concerns hydrogel implants and related methods for treating growth plate injury, particularly in pediatric patients.

### BACKGROUND

No commercially available device and surgical method exists to prevent limb deformity after physeal injury, a significant pediatric orthopaedic problem. The physis (growth plate) is the cartilaginous interfacial tissue at the ends of limb bones that drives appendicular skeletal growth. 15% of long bone fractures in children involve the physis, with a 35% prevalence in children 10 to years-old. The overall incidence of physeal injury in the juvenile population is 2.4 to 4.6 per 1,000. Up to 75% of physeal injuries cause some growth disturbance, most often from bone that forms across the physis, bridging the epiphysis, and metaphysis (boney tether). In the lower limbs, tethers cause limb deformity, length discrepancy and substantial physical impairment.

Surgical procedures are available to correct these deformities but are associated with significant disadvantages. Distraction osteogenesis is highly invasive, painful, and prolonged (3-6 months). Epiphysiodesis (hardware implantation and/or physeal bar rotation/excision) sacrifices patient height and often requires osteotomy to reshape geometry or distraction to restore length. The Langenskiold procedure (autologous transplant of fat as an interpositional material) has a high risk of bony tether recurrence (65%-82%). Moreover, surgical treatment is very costly, both monetarily and psychologically. These surgical approaches restrict activity during a child's formative years, and subject them to painful procedures, repeated clinic visits, multiple surgeries, and lengthy rehabilitation.

### SUMMARY

Disclosed herein implantable material and methods for treatment of growth plate injuries and other purposes. Exemplary compositions comprise poly (ethylene glycol) (“PEG”), gelatin (“GEL”), and heparin (“HEP”). The PEG, GEL, and HEP can comprise various forms of these materials, such as methacrylated forms, etc. The compositions can be configured to treat a growth plate injury in a patient. Some embodiments are anti-osteogenic. Some embodiments are ant-mineralization.

The disclosed materials can be in a dry power form, in a liquid form, or in a solid hydrogel form. In the liquid form, compositions can be implanted into a patient, and can then be made into a solid hydrogel form via various mechanisms, such as application of light that reacts with a photoinitiator and causes crosslinking of polymers in situ.

In some methods, the materials can be implanted as a prophylactic treatment, for example to help prevent unwanted boney tether growth while a recent injury heals. In an example, the composition can be injected into a recent growth plate fracture to help the fracture heal properly. Alternatively and/or in addition, the materials can be implanted as an interpositional implant, such as to fill a void created by removal of diseased tissue, for example. For example, a boney tether in the growth plate zone can be excised and then a hydrogel material can be implanted in the void to prevent unwanted boney tethers from growing into the void area.

In some methods, it can be beneficial to have at least part of the implanted material be positioned in the epiphyseal zone, such that the implanted material moves along with the growth plate as the bone grows.

In some embodiments, the composition can comprise various other materials, such as LAP, saline solution, cells, growth factors, drugs, and/or other components. Cells can comprise chondrocytes, stem cells, etc. Anti-osteogenic drugs included can comprise dexamethasone, recombinant sclerostin, and/or midazolam. Growth suppressing drugs included can comprise any inhibitor of mammalian target of rapamycin (“mTOR”).

In some embodiments, the compositions can be pre-loaded in a syringe, such as in a liquid or powder form. In powder form, a solution can be added prior to injection into the growth plate area of a patient.

In some embodiments, an implanted hydrogel can comprise layers that mimic growth plate zonal architecture. For example, the hydrogel can include three layers comprising a proliferative zone (“PZ”) layer, a prehypertrophic zone (“PHZ”) layer, and a hypertrophic (“HZ”) layer, with the PHZ layer between the PZ layer and the HZ layer.

The foregoing and other objects, features, and advantages of the invention will become more apparent from the following detailed description, which proceeds with reference to the accompanying figures.

### BRIEF DESCRIPTION OF THE DRAWINGS

FIGS. 1A-1C illustrate an exemplary treatment of physeal tethers (Kim HT J Korean Orthop Assoc. 2008). FIG. 1A is a T2-weighted coronal MRI of a 13 year & 8 month old boy showing the physeal bar formation following supracondylar fracture of the right distal femur. The physeal bar occupies 22% of the central area of the physis. FIG. 1B is an intraoperative fluoroscopy image showing excision of the bar using a dental burr inserted through the metaphyseal window. FIG. 1C is an image from 20 months post OP, where an external fixator was applied to correct the 3 cm leg length discrepancy and a residual genu valgum of 7 degrees.

FIGS. 2A-2D illustrate a demonstration of an exemplary injectable material (PGH) for treatment of growth plate injury. In FIG. 2A, a surgeon fills a powder-filled syringe with saline to liquify the injectable material, and then injects the liquid material into a physeal fracture or defect, simulated here with a hollow transparent tube. As shown in FIG. 2B, the injected liquid material (died pink for visualization) sets within minutes and becomes a gel. As shown in FIG. 2C, the set gel material is rigid and holding shape. In FIG. 2D, the set gel material is holding shape in a saline bath. The PGH depicted is 8% (w/v) in phosphate buffered saline with the methacrylated polymers at a ratio of 3:4:3 (poly(ethylene glycol), gelatin, and heparin).

FIGS. 3A-3C illustrate that the exemplary injectable PGH material is pro-chondrogenic and anti-osteogenic. The injected PGH material is pro-chondrogenic and anti-osteogenic/anti-mineralization due to its unique polymeric composition. FIG. 3A, illustrates three pre-polymers that can be included in the injectable material. FIG. 3B shows nuclear magnetic resonance (NMR) analysis of the molecular structure of the three pre-polymers, illustrating that the pre-polymers can be modified to make them chemically (covalently) cross-linkable. Box A shows the characteristic signal of the vinyl group at 5.7 and 6.2 ppm for methacrylated polymers. Box B shows the methacrylate signal at 1.8 ppm for methacrylated polymers. As shown in FIG. 3C, the resulting crosslinked hydrogel material is sufficiently stiff to handle as a solid, and has a stiffness (labeled “GG”) that is comparable to PEGDA (methacrylated poly(ethylene glycol)) and GEL-MA (methacrylated gelatin).

FIGS. 4A-4D are micro computed tomography (micro-CT) images of physeal defects (15 mm deep×4.5 mm diameter) created in a juvenile goat model immediately post-operation (4A, 4C) and left untreated (4B) or treated with a hydrogel material (PGH hydrogel component only, 4C, 4D). FIG. 4A is a surface rendering showing the 3 K-wires (pins, 2 spanning physis) that are used to track growth of the physis with micro-CT imaging. FIG. 4B is a CT slice showing that untreated defects lead to formation of large honey tethers across the physis after 3 months post-op (7 months age). FIG. 4C is a CT slice immediately post-OP of a defect treated with a PGH hydrogel alone, without any drugs. FIG. 4D shows the resultant inhibition of tether formation (unlike with autologous fat transplant) but lack of physeal regeneration and concomitant endochondral ossification and trabecular bone after 3 months growth (comparable to autologous fat transplant).

FIGS. 5A-5D illustrate that the exemplary injectable PGH hydrogel material is pro-chondrogenic and anti-osteogenic. FIGS. 5A and 5C show Hematoxylin & eosin (H&E) stains and FIGS. 5B and 5D show Von Kossa stains of 8% w/v hydrogels containing osteoblasts and cultured in osteogenic medium for four weeks. Scale bar=500 μm. FIGS. 5A and 5B are GEL-MA hydrogel (only methacrylated gelatin for polymeric component). FIGS. 5C and 5D are PGH hydrogel. H&E stains fibrous tissue pink and bone as dark pink, while saccharides as blue and cell nuclei as dark purple. Von Kossa stain shows presence of phosphate anions (displaces calcium ions) and background matrix as pink. The PGH hydrogel inhibits osteogenesis by osteoblasts, as evidenced by no observable mineral deposition (black) in FIG. 5D compared to GEL-MA hydrogel in FIG. 5B.

FIG. 6 shows an area for analysis of tether and regenerate tissue in treated/untreated physeal defects (yellow box). Hematoxylin and Eosin stain. (Pink=bone. Light pink=fibrous tissue. Blue=cartilage. Small pink dots with dark purple nucleus=cells.)

FIG. 7 shows a bony tether (indicated by the arrow, 100 μm width) formed between the PGH hydrogel and the physis after three months growth. Bony tethers were seen in all legs treated with experimental defects, with widths ranging from 28 um to 1665 μm. Hematoxylin and eosin staining of the physis. Hematoxylin and Eosin stain. (Pink=bone. Light pink=fibrous tissue. Dark blue=cartilage. Light purple=hydrogel. Small pink dots with dark purple nucleus=cells.)

FIG. 8 illustrates various types of growth plate fractures defined under the Salter-Harris classification system. The list is not all inclusive.

FIGS. 9A-9D are schematic representations of hydrogel insertion locations. EP=epiphysis; MP=metaphysis; Dark blue=physis (growth plate). In FIG. 9A, the hydrogel is positioned within the growth plate. In FIG. 9B, a majority of the hydrogel is positioned in the epiphysis. In FIG. 9C, a majority of the hydrogel is in the metaphysis. In FIG. 9D, part of the hydrogel is in the epiphysis and part is in the metaphysis. EP placement is shown to result in less tethering.

FIGS. 10A and 10B illustrate that the PGH hydrogels promote quiescence of stem cells in vivo. FIG. 10A shows DAPI staining (blue). “Ghost cells” are indicated by white arrows; hematoxylin positive cells are indicated by yellow arrows. FIG. 10B shows Methyl Green Pyronin staining. Methyl Green=bluish green (DNA); Pyronin Y=pink (RNA). Purple=presence of both DNA and RNA. “Ghost cells” are indicated by black arrows; cells with active transcription are indicated by red arrows.

FIGS. 11A-11D illustrate that the PGH hydrogel inhibits apatite formation as shown by lower opacity at three hours. Hydrogels were placed between supersaturated solutions of calcium and phosphate, and photographs were taken at 0 (FIG. 11A), 0.5 (FIG. 11B), 1 (FIG. 11C), and 3 hours (FIG. 11D). “C/P” indicates the calcium solution was added to the top of the transwell while “P/C” indicates the phosphate solution was added to the top. Both PGH and GEL hydrogels appeared to be more opaque after 3 hours but the PGH less so.

FIGS. 12A-12F are close-up images of the hydrogels of FIGS. 11A-11D at three hours between supersaturated solutions of calcium and phosphate. These figures illustrate that the PGH is less opaque than the GEL. “C/P” indicates the calcium solution was added to the top of the transwell while “P/C” indicates the phosphate solution was added to the top. “CTRL” is the transwell without hydrogel.

### DETAILED DESCRIPTION

Disclosed herein are materials and methods for treatment of growth plate injury, particularly in juvenile patients. These materials and methods can minimize osteogenesis and bony tether formation in the area of the growth plate, and can diminish derangement of limb growth at the physis, among other benefits.

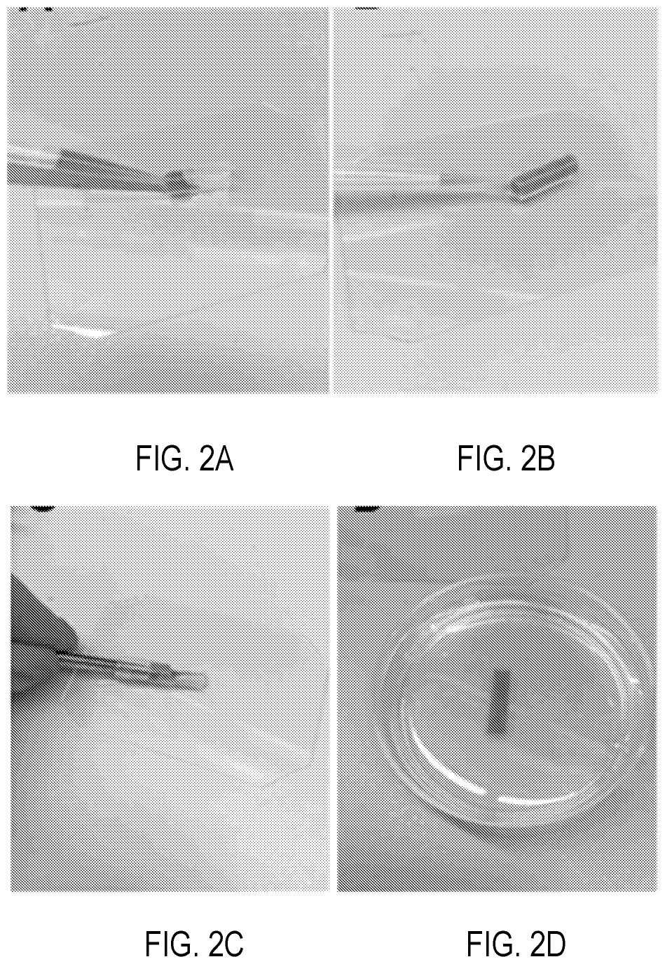

In some methods, the implantable materials can be pre-formulated and provided to a surgeon (or other healthcare provider) in liquid form for implantation into an injury site in a patient. The liquid form material can be provided in a pre-filled syringe or other container. After the liquid material is injected or otherwise placed within the intended implantation site, the liquid material can be set/solidified. FIGS. 2A-2D illustrate such an example. In FIG. 2A, an exemplary liquid material, as disclosed herein, is injected into a cylindrical container, which represents an implantation site within a patient. FIG. 2B shows the material after solidifying into a hydrogel within the cylindrical void, and FIG. 2C shows the solidified hydrogel material with the transparent shell removed, illustrating its rigidity. FIG. 2D illustrates the hydrogel material maintaining its solid form while submersed in a saline liquid.

In some cases, the materials can be provided as a dry powder contained in a syringe or other container. Such powder can be rehydrated/liquified with addition of saline or other liquid at the point-of-care, and the resulting liquid material can then be injected into the injury site. In some cases, the provided liquid or powder may be supplemented at the point of care with other components prior to implantation in a patient.

The injected/implanted liquid material can be set/solidify into a hydrogel within the patient. In some embodiments, the injected material can include a photoinitiator that causes the material to solidify when exposed to a light source. In other embodiments, the injected material can set within the patient without a photoinitiator.

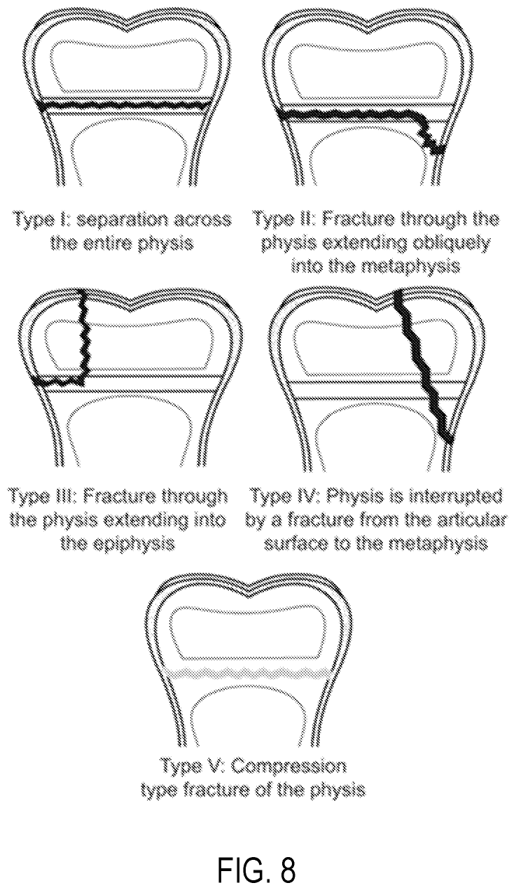

Regardless of the manner of composing the material, the manner of placing the material into the patient, and the matter of setting/solidifying the material, the resultant implanted hydrogel can be used in various situations with great benefit. In some methods, the implanted hydrogel can be used in a prophylactic manner, such as where the material is injected within a physeal fracture (e.g., see fracture types in FIG. 8), such as during reduction (closed or open), to reduce the risk of mineralization and/or bony tether formation in the fracture area while the bone and physis heal. The anti-osteogenic properties of the hydrogel inhibits formation of bony tethers that restrict expansive growth.

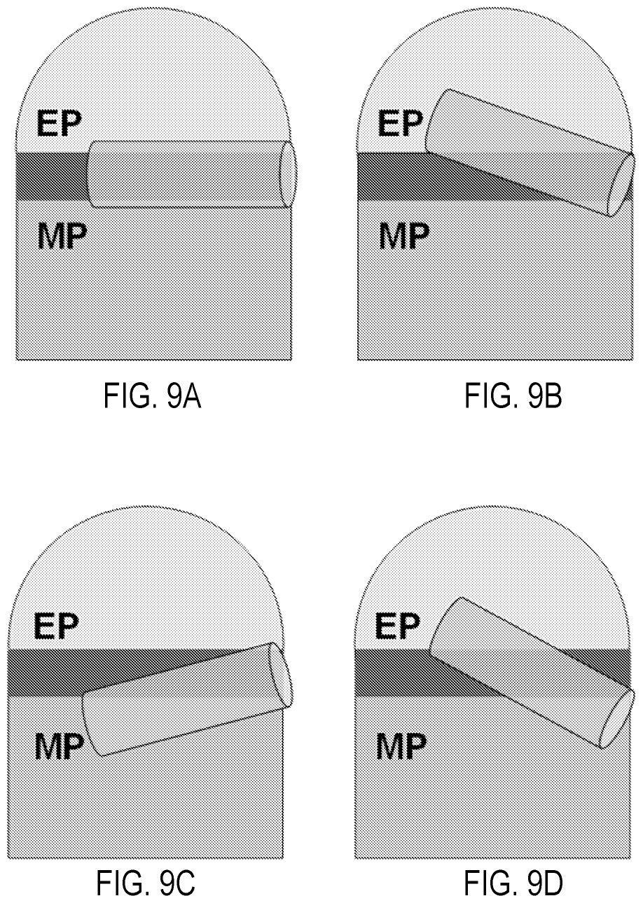

The implanted hydrogel can also be used as an interpositional implant to fill a void in the area of the growth plate, such as after resection of bony tethers, sarcoma, or other diseased physeal tissue. The implanted material can fill the void and reduce the risk of mineralization or bony tether formation in the void area. In some such embodiments, the void can be created and/or shaped such that the implant can be placed to overlay the growth plate on the epiphyseal side. For example, a surgeon can remove all boney tissue within the growth plate that bridges the epiphysis and the metaphysis. A shallow pocket can be formed in the epiphysis wider than the resection region in the growth plate. The implant can then be placed to preferably overlay the growth plate on the epiphyseal side. FIGS. 9A-9D shows exemplary placement positions of the implant relative to the growth plate, metaphasis, and epiphysis.

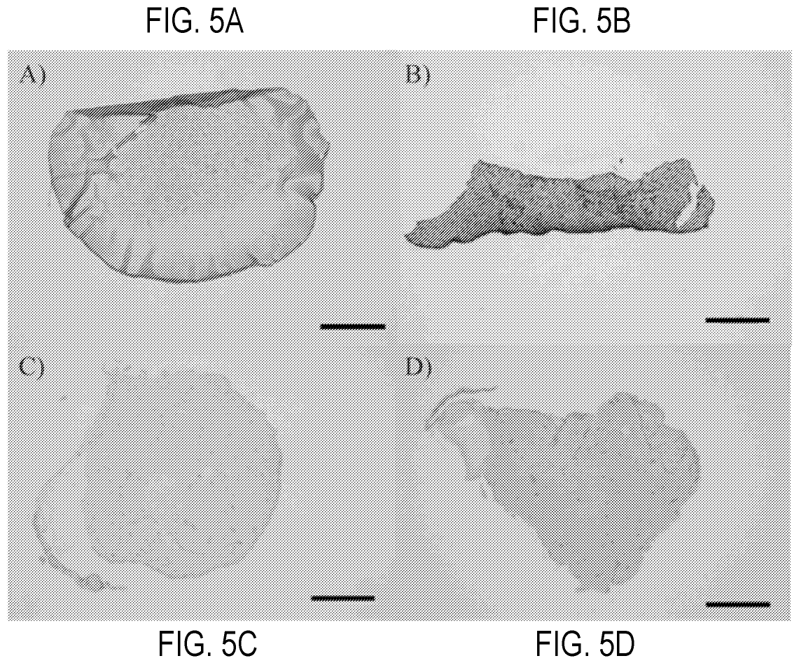

Whether implanted in a prophylactic manner, an interpositional manner, or otherwise, the implanted hydrogel can inhibit osteogenesis by progenitor cells, mineralization by hypertrophic chondrocytes, and mineralization by osteoblasts (FIGS. 5B 5D), a combination of which reduces boney tether formation. The anti-osteogenic and anti-mineralization properties of the hydrogel can be supplemented with optional added components, such as anti-osteogenic drugs, growth suppressing drugs, and/or progenitor cells and related growth factors. In addition, the hydrogel can fill the physeal fracture line and/or resection void to inhibit infiltration by vasculature and osteoprogenitors.

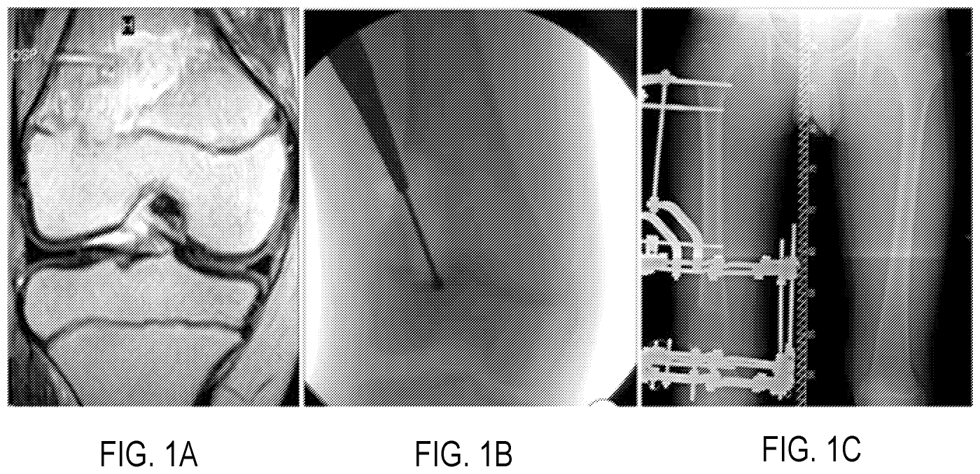

Other treatments for growth plate injury (see, e.g., FIGS. 1A-1C) have serious shortcomings compared to the herein disclosed technology. For example, they do not provide a combination of anti-mineralization and anti-osteogenic effects while still promoting cartilage growth. Surgeons currently treat epiphysiodesis with “eight-plates” and staples to restrict growth at the contralateral aspect of the physeal injury to restore limb geometry and/or restrict physeal growth at the contralateral limb to match limb length. Associated devices include distraction units used to lengthen the limb. Investigational approaches (e.g. gene therapy, cell implantation) are focused on physeal regeneration and do not inhibit boney tether formation, and none have successfully regenerated the physeal architecture and restored normal growth.

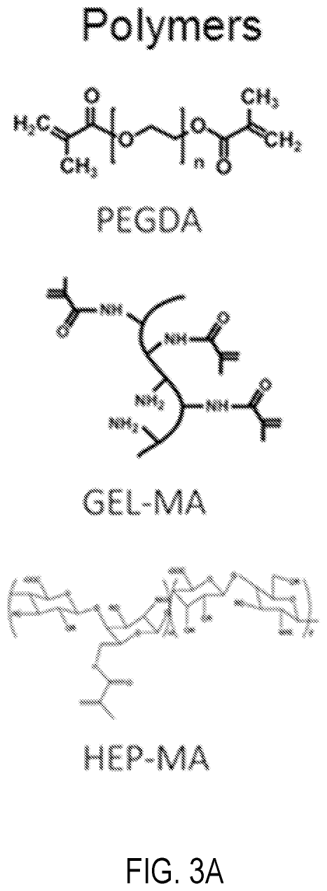

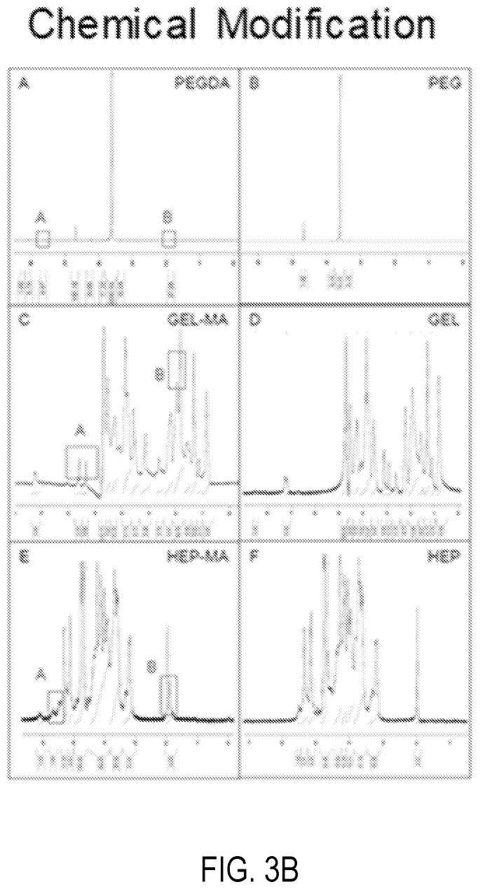

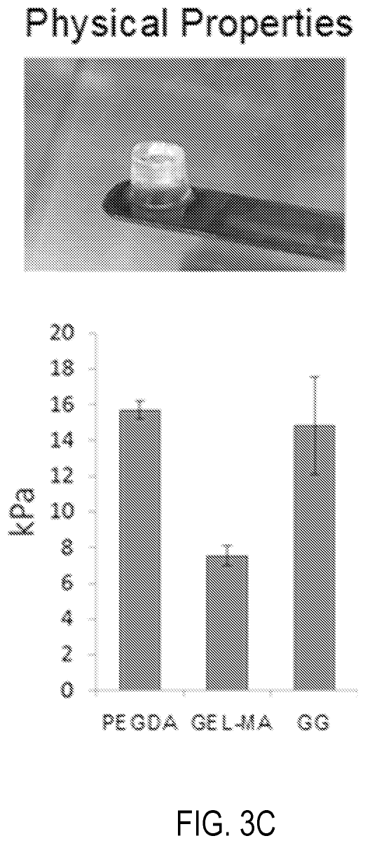

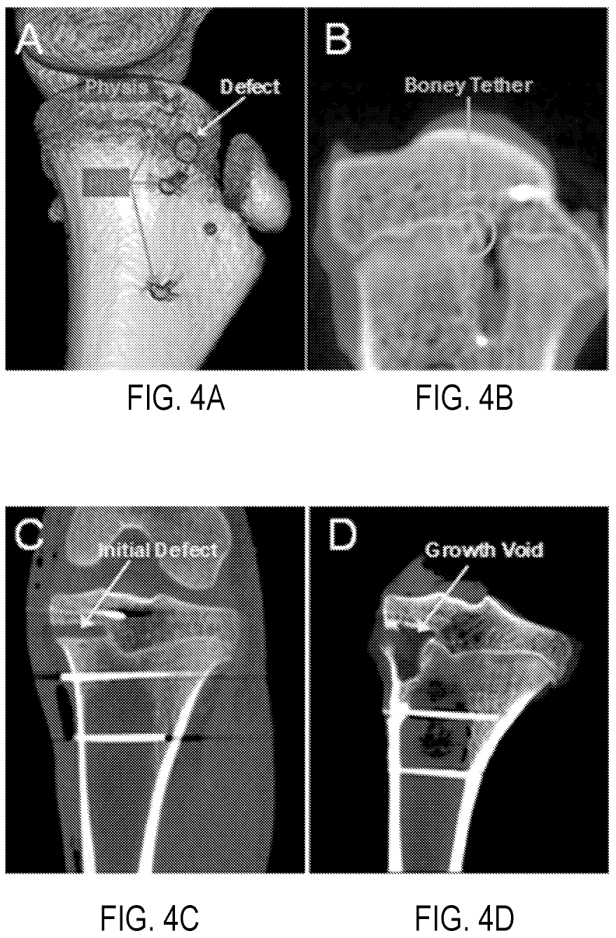

Exemplary hydrogels disclosed herein can comprise a combination of poly(ethylene glycol) (“PEG”), gelatin (“GEL”), and heparin (“HEP”). Together, this combination can be referred to as “PGH”. In some embodiments, the PGH combination can comprise methacrylated poly(ethylene glycol) (“PEGDA”), methacrylated gelatin (“GEL-MA”), and methacrylated heparin (“HEP-MA”). See FIGS. 3A and 3B. Hydrogels that include PEGDA, GEL-MA, and HEP-MA can be referred to as PGH hydrogels. Some of the hydrogels can also include a photoinitiator to provide photocrosslinkability with light, such as lithium phenyl-2,4,6-trimethylbenzoyl phosphinate (“LAP”), for example at 0.005% w/v in the hydrogel. The polymers can be rendered photo-polymerizable via addition of acryloyl/methacryloyl moieties, tyramine, styrenes. Others may include a mixture of methacrylated and thiolated polymers for crosslinkability by disulfide bond formation, thiol—ene reaction, and Michael-type addition reaction, for example a combination of PEGDA, thiolated gelatin, and HEP-MA. The physiochemical properties of the hydrogel can be modulated by adjusting the degree of thiolation and methacrylation and the fractional composition of the polymer components. The hydrogels can also include a base component, such as a saline liquid, for example phosphate buffered saline (“PBS”). FIG. 3C illustrates physical properties of an exemplary PGH hydrogel (GG).

The PEGDA can, for example, have a number average molecular weight (Me), sometimes referred to herein simply as just molecular weight or “mw”, that is equal to about 4,000 at methacrylation of 93% of terminal hydroxyl groups. The GEL-MA can, for example, have a mw=45,000 at methacrylation of near 100% of the lysine residues. The HEP-MA can, for example, have a mw=15,000 at methacrylation of 10% of available saccharide residues.

PGH hydrogels, as well as precursor compositions such as a powder preloaded in a syringe, can comprise various ratios of the constituent components PEDGA, GEL-MA, and HEP-MA. In some embodiments, the HEP-MA comprises at least 16% of a total mass of the PEGDA, the GEL-MA, and the HEP-MA combined. In some embodiments, the HEP-MA comprises at least 30% of a total mass of the PEGDA, the GEL-MA, and the HEP-MA combined. In some embodiments, the HEP-MA comprises at from 16% to 30% of a total mass of the PEGDA, the GEL-MA, and the HEP-MA combined. In some embodiments, a mass ratio of PEGDA:GEL-MA:HEP-MA is about 3:4:3. In some embodiments, a mass ratio of PEGDA:GEL-MA:HEP-MA is about 63:21:16. Some exemplary PGH hydrogels have a density of from 6% to 10% weight per volume. Some exemplary PGH hydrogels have a density of from 8% to 10% weight per volume. Some exemplary PGH hydrogels have a density of from 7.5% to 8.5% weight per volume.

In any of the embodiments disclosed herein, components of the hydrogel can be substituted with functionally analogous materials. For example, the heparin component, HEP-MA, can be substituted with chondroitin sulfate or any other highly sulfated proteoglycan. Heparin may be preferred because it is the most anionic and complexes with many growth factors due to similarity to heparan sulfate, but another highly sulfated proteoglycan may be used instead. For example, the gelatin component can be substituted with collagen. For example, the poly(ethylene glycol) component can be substituted with poly(vinyl alcohol).

The herein disclosed hydrogels can utilize any of various forms of crosslinking methods to solidify the hydrogel. In some examples, a photoinitiator such as LAP is included the hydrogel can be solidified by applying light, such as having 300-500 nm wavelengths. A number of light photo-initiating systems may be used to crosslink the polymers, including Norrish Type I and Type II and photocycloaddition systems. Other hydrogels can be physically or chemically crosslinkable (solidified) via photopolymerization, via non-photo chemical bonding (e.g., thiol-ene/thiol-Michael addition), and/or via physical reactions (e.g., hydrophilic-hydrophobic interaction). Examples of physically formed hydrogel materials include PIPAAm and poloxomer materials. In some embodiments, hydrogels can be crosslinked to form a hydrogels in situ using appropriate crosslinkers (e.g. tetrakis, genipin, transglutaminase), or via modification to provide active moieties, for example acrylated to render them crosslinkable via radicals generated with light (photocrosslinkable) and/or with persulfate salts (e.g., ammonium persulfate, potassium persulfate, sodium persulfate). Persulfate crosslinking rate can be controlled with addition of ascorbate.

More information regarding crosslinking methods, as well as compositions, formulations, uses, and other properties of hydrogels that are applicable to the technology disclosed herein, can be found in WO 2017/152112, published Jul. 26, 2018; WO 2019/183201, published Sep. 26, 2019; and WO 2019/241577, published Dec. 19, 2019, all of which are incorporated by reference herein in their entirety.

Any of the herein disclosed hydrogels or other materials can optionally also include various additional components, such as cells, growth factors, anti-osteogenic drugs, growth suppressing drugs, anti-inflammatory drugs, and/or other supplements.

Anti-osteogenic drugs can vary in structure and mechanism of actions. One type of anti-osteogenic drugs that can be included are corticosteroids and glucocorticoids, such as dexamethasone or prednisone. Dexamethasone inhibits bone formation, and a local short-term and low dose (e.g., at least 1 μM) delivery is needed for anti-tether effects using the herein disclosed technology. Another anti-osteogenic drug that can be included is sclerostin, e.g., human recombinant sclerostin. Sclerostin is a glycoprotein regulated by dexamethasone signaling that has narrow bioactivity, inhibiting bone formation and cartilage mineralization without impairing bone density and cartilage growth. Recombinant sclerostin can similarly be include in a low done (e.g., at least 1 μM). Another category of anti-osteogenic drugs that can be included are benzodiazepine derivatives, such as midazolam. Midazolam can inhibit chondrogenesis and osteogenesis by mesenchymal stem cells.

Growth suppressing drugs can include various cancer-fighting drugs. One example is Everolimus, along with other inhibitors of mammalian target of rapamycin (mTOR). Everolimus is a chemical immunosuppressant and is sometimes used in preventing organ transplant rejection and in treatment of certain tumors and cancers. It is more selective for the mTORC1 complex than the parent compound rapamycin. Inhibition of mTORC1 reduces cellular transcription and translation. The parent molecule, rapamycin, can diminish limb growth at the physis without necessarily altering mitosis, e.g. via decreased matrix synthesis and decreased chondrocyte differentiation (hypertrophy) via reduced Indian Hedge Hog secretion.

Any of the herein disclosed hydrogels or other materials can optionally also include cells, such as progenitor cells (e.g., bone marrow derived stem cells and/or chondrocytes) and related growth factors (e.g., TGFβ-1).

As noted above, the implanted PGH hydrogel can be used in a prophylactic manner, such as where the material is injected within a physeal fracture during reduction to reduce the risk of mineralization and/or bony tether formation in the fracture area while the bone heals. The cellular actions of the hydrogel include inhibition of hypertrophy by chondrocytes, osteogenic differentiation of mesenchymal progenitor cells, and mineralization by osteoblasts (cellular anti-tether mechanism). Differentiation of bone marrow derived stem cells (BMSCs) encapsulated within a hydrogel has been tested during in vitro culture and in vivo growth within subcutaneous implant pockets in mice. The hydrogel inhibited osteogenesis by goat BMSCs in vitro while permitting chondrogenesis, and enhanced chondrogenesis by human BMSCs in vivo while inhibiting mineralization compared to a gelatin bioink. Anti-mineralization, anti-osteogenic, and pro-chondrogenic effects of the PGH hydrogel arise from its unique composition of three polymers: gelatin, heparin, and poly(ethylene glycol).

Experimental data also demonstrates anti-osteogenic effects on differentiated osteoblasts (bone cells), as illustrated in FIG. 5. Mandibular condylar osteoblasts (OBs) were isolated from the goat condyles. At four weeks culture in osteogenic medium (aMEM with GlutaMAX™, 10% w/v FBS, 1X penicillin-streptomycin, 10 nM dexamethasone, 5 mM β-glycerol phosphate, and 50 μM L-ascorbic acid 2-phosphate), mandibular OBs produced mineralized matrix only in the GEL-MA hydrogel (8% w/v) and not the PGH hydrogel (8% w/v at mass ratio of 3:4:3 of PEGDA:GEL-MA:HEP-MA). Both hydrogels had similar cell number as evidenced by DNA content, but the overall DNA content was lower than at seeding.

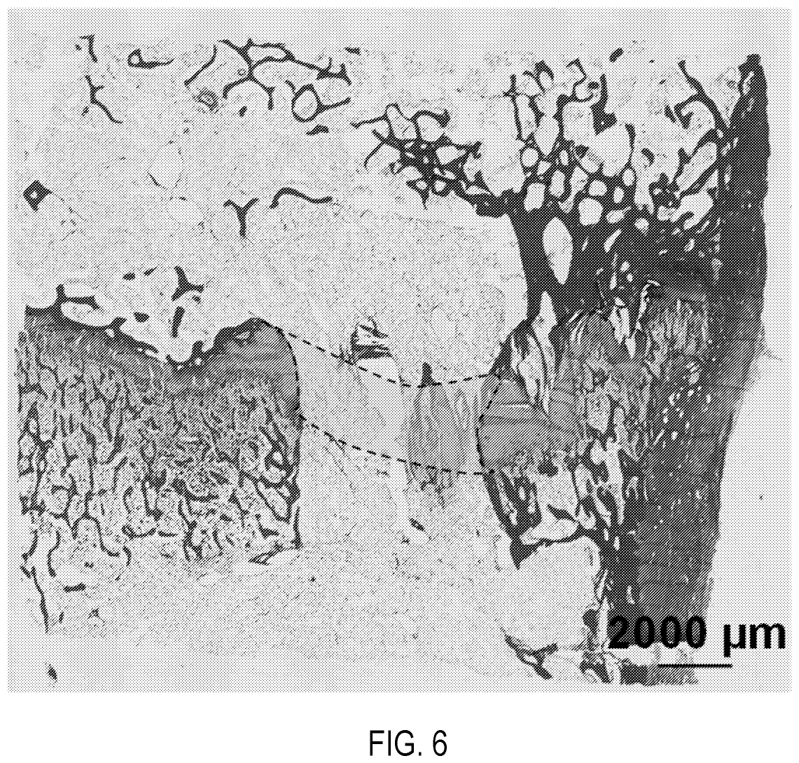

When the PGH hydrogel is used as an interpositional implant to fill a void in the area of the growth plate, the implant is preferably positioned to overlay the growth plate on the epiphyseal side, or at least such that part of the implant is on the epiphyseal side. FIGS. 9A-9D show examples of implant orientations. Placement of a cylindrical defect/implant was tested at different angles/positions relative to the proximal tibial growth plate in the juvenile goats. The tested implant was a PGH hydrogel (10% w/v) supplemented with autologous goat BMSCS (30 million/ml) and TGFβ-1 (10 μg/mL) into 4.5 mm diameter by 15 mm deep voids from the medial aspect. It was determined that placement of the implant at least partially in the epiphyseal side leads to the most inhibition of bone formation within what would have been the growth plate region and instead formation of adipose and fibrous tissue. The orientation of the defect relative to the growth plate was variable because the drilling of the defect was guided by x-ray and because the growth plate anatomy is non-planar. The defect was created in the posterior part of the medial aspect of the proximal tibial growth plate because the growth plate is most planar in that location. Therefore, the defect was skewed relative to the growth plate, allowing determination of the effect of implant placement on anti-tether efficacy. Tissue formation and limb growth were compared after 3 months to untreated defects after three months growth. The final disposition showed no bone formed within the implanted hydrogels proper. Overall, the BMSCs remained mostly in a dormant state and did not contribute to tissue formation. Measures include:
1. Quantification of regenerate tissue in the growth plate defects (see FIG. 6): The defect area was defined as that between borders of intact growth plates flanking the injury sites and of height spanning the thickness of the growth plate (note, widening of the growth plate occurred at the defect margins).
   a. Percentages of fat and bone within the defect sites were calculated as

$$\frac{Area of bone or fat}{Area of defect} \times 100 \% ( using Hematoxylin and eosin stained slides )$$

b. Percentage of the chondrogenic area was calculated as

$$\frac{Area of the chondrogenic region}{Area of hydrogel} \times 100 \% using Safranin O stained slides .$$

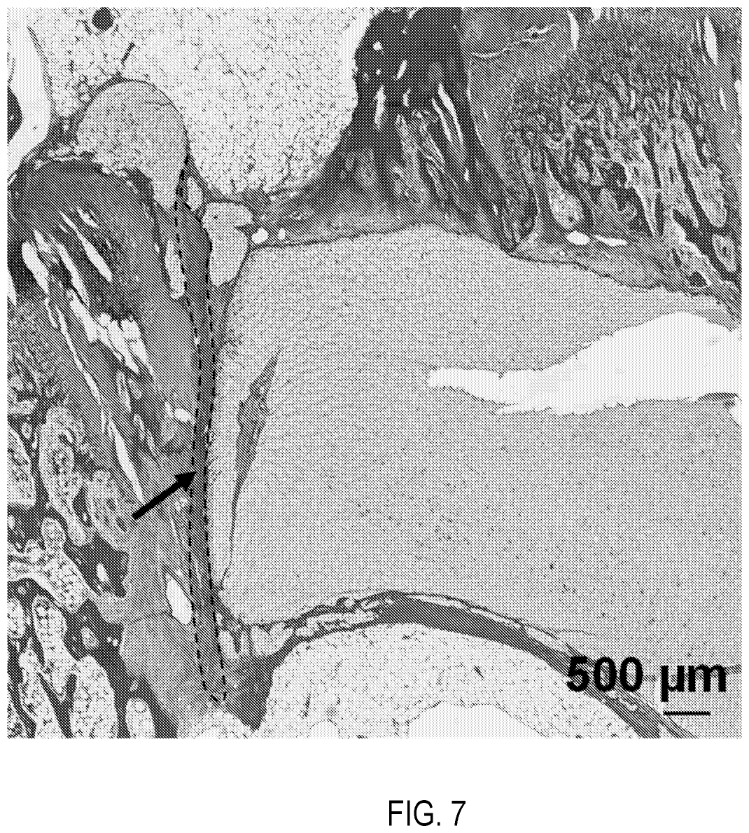

2. Tissue composition within the drill holes. The regions of interest were defined as 15 mm×2 mm rectangles within the drill holes. The drill holes were identified by landmarks such as clear boundaries of bone and fat, and residual hydrogel within cortical bones.
3. Quantity and thickness of the bony tethers along the growth plate were measured using NIS-Element AR from serial sections
   Outcomes: The implant reduced the total bone area within the physeal front. When not accounting for implant placement, the average bone area in the growth plate region (area of defect) after 3 months growth (FIG. 6) was 26%±12%, ±=standard deviation) in the hydrogel treated defects and 53%±7.0% in the untreated defects (p=0.09). The hydrogel treatment increased fat content, with 35%±17% in treated versus 3.6%±3.62% in untreated defects. However, some focal tethers were evident with hydrogel treatment, though these did not affect limb growth. As shown in FIG. 7, these formed along the interface between the hydrogel and surrounding tissue. Therefore, the effect of the surgical method was investigated and it was found that the implant approach affects success.

When accounting for implant placement, defects that had the majority of the hydrogel implanted in the epiphysis showed significantly more fat (FIG. 9B, 68%±17%) compared to the ones made primarily only within the growth plate (FIG. 9A, 39%±19%, p=0.0076) and the metaphysis (FIG. 9C, 35±26%, p=0.0241). This occurs because epiphyseal placement of the hydrogel provides for migration of the hydrogel with the physis as it grows, continuing to inhibit bone formation in the physis until the hydrogel is resorbed. Conversely, hydrogels placed within the growth plate proper with no epiphyseal superstructure to allow it (the hydrogel) to travel with the physis are left behind and can no longer function to restrict tether formation. Of note,
Growth in this goat model is very rapid compared to human children. The hydrogel as interpositional material will perform better in humans as the interface between the hydrogel and surrounding defect margin is maintained longer with the slower growth, better inhibiting angiogenesis and migration of progenitor cells and osteoblasts into the space between the two where tethers are formed (see FIG. 7).
The hydrogel resorbs faster in the epiphysis. The percent area occupied by hydrogel within the defect hole was significantly higher when in the metaphysis (27±31%) compared to in the epiphysis (0%, p=0.0001) and right at the growth plate (1.4±3.8%, p<0.0001).
The PGH hydrogels did not mineralize even when encapsulated in a bony environment (e.g., cortical bone), confirmed by CT images.

The PGH hydrogels disclosed herein can also be considered “anti-mineralization,” and can have the anti-mineralization properties such as promoting quiescence of stem cells, inhibiting osteogenic activity of osteoblasts, and effect on ion transport and mineral formation sans cells.

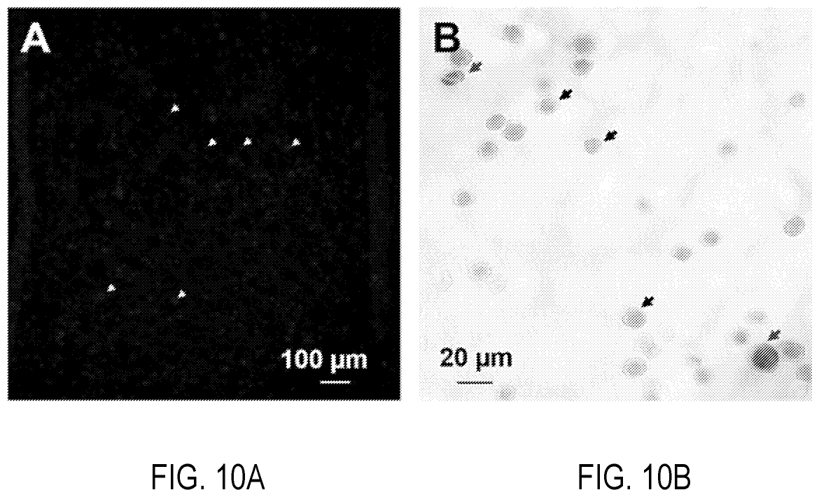

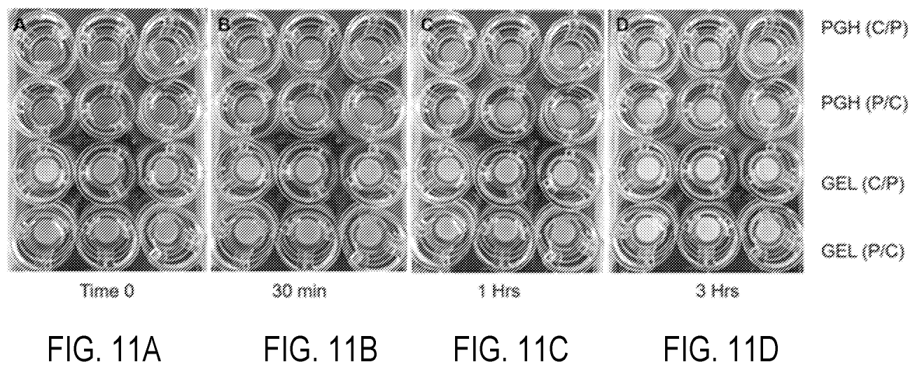

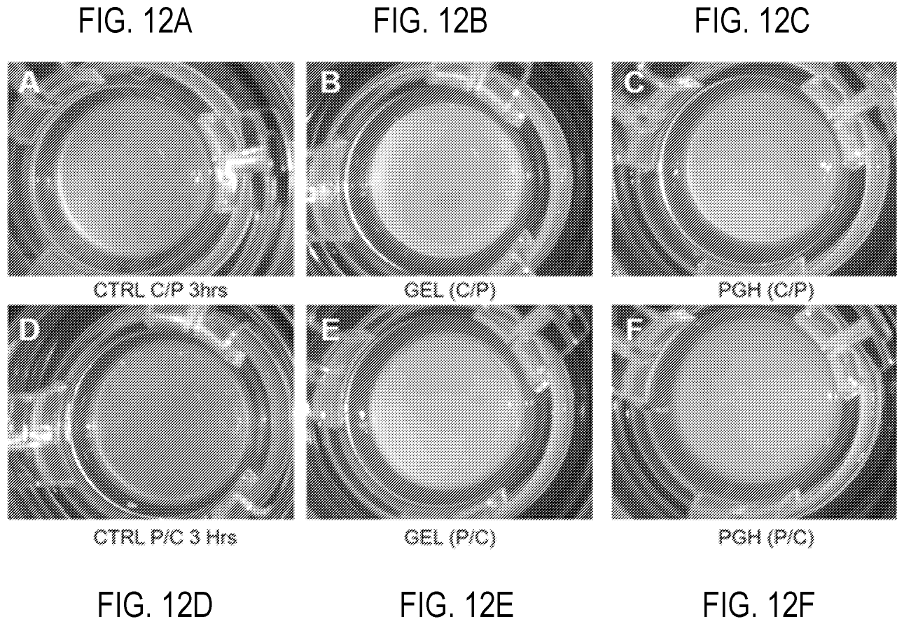

Only a few hydrogel implants showed chondrogenesis by the encapsulated goat BMSCs. Interestingly, the non-chondrogenic cells in these hydrogels can be categorized into two types: one had nucleus stained dark purple by hematoxylin, often found near proteoglycan producing BMSC derived chondrocytes; the other was not stained by hematoxylin, but Fast Green or eosin only. These “ghost cells” were seen in all remnant hydrogels, distributed throughout the scaffold, regardless of whether chondrogenesis occurred or not. DAPI stain showed that the “ghost cells” had nuclei, though the stain was much fainter compared to the hematoxylin positive cells (FIG. 10A). To further investigate the identity of the “ghost cells,” they were stained by Methyl Green Pyronin. The results showed that these cells had DNA, but RNA was undetectable (FIG. 10B). Hematoxylin positive staining cells stained more strongly with DAPI than the hematoxylin negative cells, suggesting the chromatin might be packed differently between the two cell types, resulting in different dye-binding affinities. Lack of hematoxylin and Pyronin Y stains indicated that these cells contained an extremely low level of RNA, a characteristic of quiescent stem cells. Quiescence can enable lasting preservation of stem cells at the target site for regeneration when necessary. The PGH hydrogel also can have an effect on ion transport and mineral formation sans cells. A calcium phosphate crystallization assay was constructed in the hydrogels by placing hydrogels (no cells) between supersaturated solutions of calcium and phosphate. GEL-MA (10% w/v) hydrogel and PGH hydrogel (10% w/v, 63:21:16 of methacrylated PEG, gelatin, and heparin) were cast in transwells (12 well dish, 1 cm² culture area, 3 μm pore size). 10x PBS (pH 7.4, 67 mM PO4) was added to one side of the well, and 100 mM CaCl₂ solution was added to the other (bottom well=2 mL, top well=500 μL solution). Photographs were taken at 0, 0.5, 1, and 3 hours to monitor apatite formation. Compared to GEL-MA hydrogels, PGH hydrogels were less permissive for mineral deposition as indicated by the slower development of opacity over time (FIGS. 11 and 12). It should be noted that this concentration of ion was supraphysiologic. No visible mineralization occurred in PGH hydrogel at physiologic concentrations by 3 hours.

For purposes of this description, certain aspects, advantages, and novel features of the embodiments of this disclosure are described herein. The disclosed methods, apparatuses, and systems should not be construed as limiting in any way. Instead, the present disclosure is directed toward all novel and nonobvious features and aspects of the various disclosed embodiments, alone and in various combinations and sub-combinations with one another. The methods, apparatuses, and systems are not limited to any specific aspect or feature or combination thereof, nor do the disclosed embodiments require that any one or more specific advantages be present or problems be solved.

Features, integers, characteristics, or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

Although the operations of some of the disclosed methods are described in a particular, sequential order for convenient presentation, it should be understood that this manner of description encompasses rearrangement, unless a particular ordering is required by specific language. For example, operations described sequentially may in some cases be rearranged or performed concurrently. Moreover, for the sake of simplicity, the attached figures may not show the various ways in which the disclosed methods can be used in conjunction with other methods.

As used herein, the terms “a”, “an”, and “at least one” encompass one or more of the specified element. That is, if two of a particular element are present, one of these elements is also present and thus “an” element is present. The terms “a plurality of” and “plural” mean two or more of the specified element. As used herein, the term “and/or” used between the last two of a list of elements means any one or more of the listed elements. For example, the phrase “A, B, and/or C” means “A”, “B,”, “C”, “A and B”, “A and C”, “B and C”, or “A, B, and C.” As used herein, the term “coupled” generally means physically, chemically, electrically, magnetically, or otherwise coupled or linked and does not exclude the presence of intermediate elements between the coupled items absent specific contrary language.

In view of the many possible embodiments to which the principles of the disclosed technology may be applied, it should be recognized that the illustrated embodiments are only examples and should not be taken as limiting the scope of the disclosure. Rather, the scope of the disclosure is at least as broad as the following claims. We therefore claim all that comes within the scope of these claims and their equivalents.

1. A composition comprising:
poly (ethylene glycol) (“PEG”);
gelatin (“GEL”); and
heparin (“HEP”).

2. The composition of claim 1, wherein the composition is anti-osteogenic, anti-mineralization, and/or configured to treat a growth plate injury in a patient.

3.-4. (canceled)

5. The composition of claim 1, wherein the HEP comprises at least 16%, at least 30%, or from 16% to 30%, of a total mass of the PEG, the GEL, and the HEP combined.

6.-7. (canceled)

8. The composition of claim 1, wherein a mass ratio of PEG:GEL:HEP is about 3:4:3 or about 63:21:16.

9. (canceled)

10. The composition of claim 1, wherein the composition is a powder, a liquid, and/or comprises a crosslinked or crosslinkable hydrogel.

11.-12. (canceled)

13. The composition of claim 1, wherein the composition comprises a crosslinked or crosslinkable hydrogel with a density of 6% to 10% w/v or 8% to 10% w/v.

14. (canceled)

15. The composition of claim 1, wherein the composition further comprises a photoinitiator.

16. The composition of claim 15, wherein the photoinitiator comprises lithium phenyl-2,4,6-trimethylbenzoylphosphinate (“LAP”).

17. The composition of claim 1, wherein the composition further comprises phosphate buffered saline, mesenchymal stem cells, chondrocytes, a growth factor, an anti-osteogenic drug, and/or a growth-suppressing drug.

18.-20. (canceled)

21. The composition of claim 201, wherein the composition further growth factor comprises transforming growth factor beta-1 (“TGFβ-1”).

22. (canceled)

23. The composition of claim 221, wherein the composition further comprises dexamethasone, recombinant sclerostin, and/or midazolam.

24. (canceled)

25. The composition of claim 1, wherein the composition further comprises an inhibitor of mammalian target of rapamycin (“mTOR”).

26. The composition of claim 1, wherein the HEP is substituted with a highly sulfated proteoglycan.

27. A syringe containing a powder, the powder comprising the composition of claim 1.

28. The syringe of claim 27, wherein the powder is configured to form an injectable liquid with addition of a saline liquid (e.g., PBS) into the syringe, and/or wherein the syringe is configured to inject the composition into a site of a growth plate injury in a patient.

29. (canceled)

30. A hydrogel comprising the composition of claim 1.

31. The hydrogel of claim 30, wherein the hydrogel comprises three layers that mimic growth plate zonal architecture.

32. The hydrogel of claim 31, wherein each of the three layers comprises chondrocytes, and/or
the three layers comprise proliferative zone (“PZ”) layer, a prehypertrophic zone (“PHZ”) layer, and a hypertrophic (“HZ”) layer, with the PHZ layer between the PZ layer and the HZ layer.

33. (canceled)

34. The hydrogel of claim 32, wherein the PZ layer comprises parathyroid hormone (“PTH”) and/or the HZ layer comprises triiodo-L-thyronine (“T3”).

35. (canceled)

36. A method comprising:
combining the composition of claim 1 with a saline liquid and a photoinitiator or other catalyst to form an implantable material.

37. The method of claim 36, further comprising implanting the implantable material at a site of a growth plate injury in a patient.

38. The method of claim 37, wherein the implantable material is injected with a syringe into the site of the growth plate injury.

39. The method of claim 37, wherein the implantable material is implanted into the site of a growth plate fracture as a prophylactic treatment.

40. The method of claim 37, wherein the implantable material is implanted into a void adjacent the growth plate after resection of bony tether, sarcoma, or diseased physeal tissue as an interpositional material.

41. The method of claim 36, further comprising applying light to the implantable material to transform the implantable material into a hydrogel.

42. The method of claim 41, wherein the application of light occurs while the implantable material is within a patient.

43. The method of claim 37, wherein the implantable material is implanted to overlay a growth plate of a bone with a majority of the implantable material in a epiphysis of the bone, such that the implanted material travels with the physis as the bone grows over time.
